(19) United States
(12) Patent Application Publication
(10) Pub. No.: US 2025/0127748 A1
(43) Pub. Date: Apr. 24, 2025

(54) METHODS FOR THE TREATMENT OF MIGRAINE AND RELATED HEADACHE SYMPTOMS USING TRICAPRYLIN

(71) Applicant: CERECIN INC., Denver, CO (US)

(72) Inventors: Samuel T. Henderson, Golden, CO (US); Judith Walker, Edinburgh (GB)

(21) Appl. No.: 18/683,891

(22) PCT Filed: Aug. 15, 2022
(86) PCT No.: PCT/US2022/040340

§ 371 Date: Feb. 15, 2024

Related U.S. Application Data
(60) Provisional application No. 63/351,684, filed on Jun. 13, 2022.
(60) Provisional application No. 63/343,893, filed on May 19, 2022.
(60) Provisional application No. 63/233,552, filed on Aug. 16, 2021.

(51) Int. Cl. A61K 31/23; A61K 47/10; A61K 47/14; A61K 47/22; A61K 47/24; A61P 25/06

(52) U.S. Cl. CPC A61K 31/23; A61K 47/10; A61K 47/14; A61K 47/22; A61K 47/24; A61P 25/06

(57) ABSTRACT

The present disclosure relates to methods for the treatment or prevention of headache, such as migraine and related headache. The methods include administering compositions comprising at least one compound capable of elevating ketone body concentrations in a subject in need thereof (e.g., ketogenic compounds), administered in an amount effective for treatment or prevention of headache, such as migraine and related headache. In one embodiment, the composition includes medium chain triglycerides (MCT), such as tricaprylin.

### CROSS REFERENCE TO RELATED APPLICATIONS

This application claims the benefit of U.S. Provisional Application No. 63/233,552, filed Aug. 16, 2021, U.S. Provisional Application No. 63/343,893, filed May 19, 2022, and U.S. Provisional Application No. 63/351,684, filed Jun. 13, 2022, each of the disclosures of which is hereby incorporated by reference in its entirety.

### FIELD OF THE INVENTION

The invention pertains to the field of treatment and prevention of migraine. More particularly, the invention pertains to the use of ketogenic agents to treat and prevent the occurrence of migraine headaches.

### BACKGROUND OF THE INVENTION

Migraine is a highly prevalent, disabling, and costly neurological disorder. Its episodic form is characterized by recurrent (0 to 14 headache days per month) moderate to severe headache attacks that last between 4-72 hours. The headaches are aggravated by physical activity and accompanied by either photo-, phono-, or osmophobia, nausea, or a combination of these. Migraine has a peak incidence in young adults during the most productive years of life, and inflicts a substantial cost on society. Current migraine treatment options have generally limited efficacy and often have side-effects.

Migraine's primary pathogenic mechanisms are still largely unknown, yet ketogenic diets have shown some limited efficacy in treating migraine. Ketogenic diets (KD) were introduced in 1921 for the treatment of drug resistant epilepsy. Ketogenic diets are high fat low carbohydrate, low protein diets that mimic fasting and induce a state of ketosis in subjects. Ketogenic diets have also been shown to be beneficial for weight loss and shown potential in other neurological conditions.

The first known report of KD in migraine appears to be in 1928 by Schnabel who tested a KD in 23 migraine patients. Nine of the 23 patients reported some improvement, however of those whose attacks recurred three confessed to breaking the diet and urine failed to detect ketosis. The authors concluded that “It would seem to be a difficult matter for adults to remain on a ketogenic diet judging from our group of patients” and that they were “sufficiently encouraged to continue with the high fat method”.

In 2010 Kossoff and co-authors examined the efficacy of a modified Atkin's diet on adolescents with frequent headaches. The modified Atkins diet induces ketosis similar to a traditional ketogenic diet, but does not impose protein restriction. The study examined 8 subjects with a minimum of 15 headaches per week for at least 3 month's duration. Three of the subjects reported improvement in headache severity and quality of life but continued to have daily migraines and required pharmacological therapy. The authors concluded “There does not appear to be any clear benefit for the modified Atkins diet in the treatment of adolescents with chronic daily headache. In addition, had improvement even been noted in this pilot study, this diet was perceived as too restrictive and most adolescents refused to try”.

In 2013 Di Lorenzo et al reported on two twin sisters who had each begun a KD for weight loss. The sisters also suffered from high frequency migraines, each having 5-6 attacks per month. The patients described severe throbbing headaches of up to 72 hours' duration. The severity was increased by movement and the attacks were accompanied by photophonophobia, nausea, and occasionally vomiting. The patients kept diligent migraine diaries and the migraines were never considered chronic (>15 month) and they never met the criteria for medication overuse headache (MOH), a clinical condition in which the high number of symptomatic analgesics taken to treat headache attacks turns into the cause of pain. The patients were also overweight, with BMIs of 28.65 and 26.81. To lose weight they began a supervised weight loss KD using meal replacement products produced by SDM Genola consisting of <1 g/kg/day carbohydrates; 1.2-1.6 g/kg/day proteins) comprising three daily meals plus one daily meal of meat or fish. The patients followed this diet for 4 weeks, then switched to a low calorie, non-ketogenic, low carbohydrate diet for two months. This cycle was repeated 3 times until desired BMI was reached (˜21). In both cases the patients reported disappearance of the migraines during periods of the ketogenic diet and reoccurrence during the transitional diets.

In the same report the Di Lorenzo et al 2013 authors describe preliminary data presented at the Italian Society for the Study of Headaches. In this study 108 migraineurs (52 treated with KD and 56 with a low calorie diet seems to indicate “that that ketogenesis and not weight loss improves migraine: there was a very high responder rate (about 90%) during a one-month KD, while two months after the four-week period of ketogenesis the KD group did not differ from the standard diet group in terms of headache reduction.”

In a follow up study Di Lorenzo et al 2015 enrolled 96 overweight female migraineurs in diet clinic to receive either KD for 1 month followed by a low-calorie standard diet (SD) or the SD for 6 months. Mean monthly attack frequency, number of days with headaches and tablet intake were assessed before and 1, 2, 3 and 6 months after diet initiation. In the KD group, the baseline attack frequency (2.9 attacks per month), number of days with headaches (5.11 days per month) and tablet intake (4.91 doses per month) were significantly reduced after the first month of diet (respectively 0.71, 0.91, 0.51; overall, KD versus baseline, P<0.0001).

During the transition period (first versus second month), the KD group showed a transient worsening of each clinical headache variable (respectively 2.60, 3.60, 3.07), despite being improved compared with baseline, with continuous improvement up to month 6 (respectively 2.16, 2.78, 3.71).

In the SD group, significant decreases in the number of days with headaches and tablet intake were observed only from month 3 (P<0.0001), and in attack frequency at month 6 (P<0.0001).

In 2015 Bracaglia et al investigated the potential mechanism responsible for ketogenic diet effectiveness in migraine. The authors recorded visual (VEPs) and somatosensory (SSEPs) evoked-potentials before and after 1-month intervention with KD in 16 migraineurs. After 1-month of KD, a significant reduction of mean migraine frequency (from a mean of 4.1 to 1.4 attacks/month, p<0.001) and duration (from 51.9 to 16.3 hours/month, p<0.001) was observed.

KD did not change 1st SSEP and VEP block of 100 sweeps, but significantly induced normalization of the interictally reduced VEPs (from +0.09 to −0.14, p=0.017) and SSEPs (from 0.38 to −0.48, p=0.002) habituation during the subsequent blocks.

The authors concluded “We found evidence for KD-induced changes at cortical level in parallel to an improvement of migraine. Since KD was able to restore normal EPs habituation curves during stimulus repetition without changing significantly early amplitude responses, we hypothesize that KD acts on habituation via an enhancement of late GABA inhibition.”

In a further study Di Lorenzo et all 2018 examined the effects of a three-month modified Atkins diet (MAD) in drug resistant cluster headache. Eighteen drug resistant cluster headache patients were put on MAD for 12 weeks. Of the 18 patients, 15 were considered responders, with 11 experiencing full resolution of headaches. The mean monthly number of headaches reduced from 108.71 to 31.44 at the end of 12 weeks.

The mechanism of action (MOA) for reduction in migraines and cluster headaches by KD has not be fully elucidated. However, several authors have speculated on the MOA and focused on two main pathways, dopaminergic stimulation and increase in GABAergic activity.

Di Lorenzo et al 2018 speculated on two mechanisms that mediate improvement in migraines and cluster headaches. First, KD can induce an increase in brain dopaminergic activity in mice and that this corrects impaired dopaminergic stimulation associated with cluster headaches and migraines. Second, ketone bodies can increase GABAergic activity in rat brains, and that the GHB is a GABA agonist an has been shown to prevent CH attacks. GABA and glutamic acid are the main inhibitory and excitatory neurotransmitters of central nervous system. Among other functions they modulate the pain threshold in the CNS. For this reason, it has been hypothesized that anomalies of GABA and glutamate turn-over may play a role in migraine pathogenesis. Additional MOAs are also possible including reduction of inflammation, inhibition of oxidative stress, improvement in ATP levels, and improvement in cerebral blood flow.

US 2018/0200220 A1 discloses the use of ketogenic medium chain triglycerides (KMCT) as follows: “The present inventors have surprisingly discovered that providing ketogenic medium chain triglycerides (KMCT) to patients suffering from frequent migraine attacks can reduce frequency of migraine onset and reduce migraine symptoms, without the side effects of medications currently used for this purpose. KMCT are metabolized in the liver to provide a rich source of ketone bodies, which can be metabolized as a carbon and energy source for the body, especially the brain.” However, this application is limited to speculation as to the efficacy of the disclosed mixtures of KMCTs.

Further research and treatment options related to the use of the ketogenic diet and ketogenic agents is needed.

### SUMMARY OF THE INVENTION

In one aspect, the disclosure relates to a method for the treatment or prevention of headache in a subject in need thereof, the method comprising administering an effective amount of a composition comprising at least 95% pure tricaprylin to a subject in need thereof.

In certain aspects, the methods are for the treatment or prevention of migraine and related headaches (e.g., trigeminal autonomic cephalgia). In some embodiments, the method is for the treatment or prevention of chronic migraine in a subject, wherein the subject experiences at least 15 migraine headaches in a consecutive 30 day period at baseline.

In accordance with the methods of the disclosure, treatment or prevention may include treatment, prevention or amelioration of one or more symptoms of headache, reduction of headache days, reduction of use of headache medication use, etc.

In some embodiments, the method provides reduction of at least 1, at least 2, at least 2.5, at least 3, at least 3.5, at least 4, at least 4.5, at least 5, at least 6, at least 7, at least 8, at least 9, at least 10, at least 11, at least 12, at least 13, at least 14, at least 15 migraine headache days a month (consecutive 30 days) after treatment for at least 1 month, at least 2 months, at least 3 months, etc.).

In other embodiments, the method provides for improvement or amelioration in one or more symptoms of migraine selected from: change from baseline in duration of migraine during treatment months 1, 2 or 3; change from baseline in duration of migraine of moderate to severe intensity during treatment months 1, 2 or 3; the proportion of participants with a 50% reduction from baseline in number of migraine headache days in treatment months 1, 2, or 3; the proportion of participants with a 75% reduction from baseline in number of migraine headache days in treatment months 1, 2, or 3; the proportion of participants with a 90% reduction from baseline in number of migraine headache days in treatment months 1, 2, or 3; change from baseline in monthly acute migraine medicine use during treatment months 1, 2 or 3; change from baseline to time to first usage of acute migraine medication during treatment months 1, 2, or 3; change from baseline Headache Impact Test (HIT-6) score (see e.g., Yang M, Rendas-Baum R, Varon S F, Kosinski M. Validation of the Headache Impact Test (HIT-6™) across episodic and chronic migraine. Cephalalgia. 2011 February; 31(3):357-67, the disclosure of which are incorporated herein in its entirety) at the end of months 1, 2 or 3; occurrence migraine associated adverse events including aura, nausea, vomiting.

In some aspects, the tricaprylin is administered in an amount ranging from about 10 g/kg/day to about 60 g/kg/day of compound in the composition, administered orally in single or divided doses. In some embodiments, the composition is administered once, twice, or three times daily.

In some aspects, the composition comprises at least about 30% by weight of the total composition of tricaprylin, and one or more emulsion forming excipients present at a concentration sufficient to form an emulsion that is stable for at least one month under ambient conditions.

In some embodiments, the one or more emulsion forming excipients of the composition are selected from the group consisting of lecithin, hydrogenated castor oils, caprylate esters, sodium oleate, glycerol, citric acid esters of monoglycerides and diglycerides, monoglycerides and diglycerides of fatty acids including propylene glycol monocaprylate, and combinations thereof

In some embodiments, the tricaprylin is present in the composition in an amount of between about 30% and about 60% by weight of the total composition. In some embodiments, the one or more emulsion forming excipients are present in the composition in an amount of between about 1% and about 10% by weight of the total composition. In some embodiments, the stable emulsions exhibits an average particle diameter of less than 0.5 μm for at least one month at ambient conditions, preferable less than 0.3 μm for at least one month at ambient conditions, preferable less than 0.2 μm for at least one month at ambient conditions.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the detailed descriptions are to be regarded as illustrative in nature and not restrictive.

### BRIEF DESCRIPTION OF THE DRAWINGS

FIG. 1 illustrates an exemplary dosing and titration scheme according to a clinical protocol of the disclosure.

FIG. 2 illustrates baseline demographics (FAS), showing mostly balanced between treatment arms; tricaprylin arm had higher proportion of men, in accordance with embodiments of the disclosure.

FIG. 3 illustrates baseline demographics (FAS), showing tricaprylin arm had a higher mean weight, though BMI was similar, in accordance with embodiments of the disclosure.

FIG. 4 illustrates baseline number of migraine headache days, showing higher proportion of subjects in the tricaprylin arm with chronic migraine, in accordance with embodiments of the disclosure.

FIG. 5 illustrates primary efficacy analysis: intended titration evaluable for efficacy set (EEITS), showing efficacy at Month 2, positive trend at Month 3, in accordance with embodiments of the disclosure.

FIG. 6 illustrates primary efficacy analysis: EEITS subgroup with 4-24 MHDs at baseline, in accordance with embodiments of the disclosure.

FIG. 7 illustrates primary endpoint: results of MMRM analyses in EES and EEITS, in accordance with embodiments of the disclosure.

FIG. 8A-8B illustrates primary endpoint: change from baseline in episodic (FIG. 8A) and chronic (FIG. 8B) subjects (EES 4-24 MHDs), in accordance with embodiments of the disclosure.

FIG. 9 illustrates secondary endpoint: change from baseline in acute migraine medication use (EES), in accordance with embodiments of the disclosure.

FIG. 10 illustrates secondary endpoint: change from baseline in acute migraine medication use (EEITS), in accordance with embodiments of the disclosure.

FIG. 11 illustrates secondary endpoints: responder analysis MHD50, MHD75, MHD90 (EES), in accordance with embodiments of the disclosure.

FIG. 12 illustrates secondary endpoints: responder analysis MHD50, MHD75, MHD90 (EEITS), in accordance with embodiments of the disclosure.

### DETAILED DESCRIPTION OF THE INVENTION

In one aspect, the present disclosure relates to methods for the treatment or prevention of headache in a subject in need thereof, the method comprising: administering an effective amount of a composition comprising a compound capable of elevating ketone body concentrations in the body of a subject in need thereof.

In some embodiments, the methods are for the treatment or prevention of migraine or related headaches (e.g., trigeminal autonomic cephalgia).

In some embodiments, the method is for the treatment of chronic migraine in a subject, wherein the subject experiences at least 15 migraine headaches (or more than 14 migraine headaches) in a consecutive 30 day period at baseline.

In accordance with the methods of the disclosure, treatment or prevention may include treatment, prevention or amelioration of one or more symptoms of headache, reduction of headache days, reduction of use of headache medication use, etc.

In some embodiments, the method provides reduction of at least 1, at least 2, at least 2.5, at least 3, at least 3.5, at least 4, at least 4.5, at least 5, at least 6, at least 7, at least 8, at least 9, at least 10, at least 11, at least 12, at least 13, at least 14, at least 15 migraine headache days a month (consecutive 30 days) after treatment for at least 1 month, at least 2 months, at least 3 months, etc.

In some embodiments, the method provides for improvement or amelioration in one or more symptoms of migraine selected from: change from baseline in duration of migraine during treatment months 1, 2 or 3; change from baseline in duration of migraine of moderate to severe intensity during treatment months 1, 2 or 3; the proportion of participants with a 50% reduction from baseline in number of migraine headache days in treatment months 1, 2, or 3; the proportion of participants with a 75% reduction from baseline in number of migraine headache days in treatment months 1, 2, or 3; the proportion of participants with a 90% reduction from baseline in number of migraine headache days in treatment months 1, 2, or 3; change from baseline in monthly acute migraine medicine use during treatment months 1, 2 or 3; change from baseline to time to first usage of acute migraine medication during treatment months 1, 2, or 3; change from baseline Headache Impact Test (HIT-6) score at the end of months 1, 2 or 3; occurrence migraine associated adverse events including aura, nausea, vomiting, etc.

In some embodiments, the compositions useful in connection with the methods of disclosure may comprise tricaprylin. Tricaprylin belongs to a family of compounds known as medium chain triglycerides (MCTs). MCTs are triacylglycerols where the fatty acids are 6-12 carbons in length. In the case of tricaprylin, greater than 95%, 97%, 98%, 99%, etc. of the fatty acids are octanoic acid comprised of 8 carbons (C8). The remaining fatty acids may be C6, C10, etc. In certain embodiments, less than 5%, 4%, 3%, 2% or 1% of the fatty acids are C10.

The MCTs of the disclosure may be prepared by any process known in the art, such as direct esterification, rearrangement, fractionation, transesterification, or the like. Sources of the MCT include any suitable source, semi-synthetic, synthetic or natural. Exemplary sources of tricaprylin include MIGLYOL® 808 or NEOBEE® 895. Examples of natural sources of MCT include plant sources such as coconuts and coconut oil, palm kernels and palm kernel oils, and animal sources such as milk from any of a variety of species, e.g., goats. For example, the lipids may be prepared by the rearrangement of a vegetable oil such as coconut oil. The length and distribution of the chain length may vary depending on the source oil. For example, MCT containing 1-10% C6, 30-60% C8, 30-60% C10, 1-10% C10 are commonly derived from palm and coconut oils. Exemplary sources of tricaprylin include MIGLYOL® 808 or NEOBEE® 895.

In certain embodiments, the tricaprylin may be administered as pharmaceutical compositions. In certain aspects, the tricaprylin may be administered as pharmaceutical composition comprising a high drug loading of tricaprylin and at least one surfactant. In some embodiments, the pharmaceutical composition may comprise a high drug loading of tricaprylin and at least two surfactants. In some embodiments, the pharmaceutical composition forms an emulsion that is stable for at least about 4 hours at ambient conditions, at least 24 hours at ambient conditions, at least 2 weeks at ambient conditions, at least one month at ambient conditions, etc.

In certain embodiments, the emulsions may have a mean droplet diameter of less than about 1000 nm, but greater than about 100 nm, e.g. between about 100 nm and 500 nm, between about 200 nm and about 300 nm, between about 160 nm and about 190 nm, etc. In some embodiments, the stable emulsions exhibit an average particle diameter of less than 0.5 μm for at least one month at ambient conditions, preferable less than 0.3 μm for at least one month at ambient conditions, preferable less than 0.2 μm for at least one month at ambient conditions. In other aspects, the emulsions may have a mean particle diameter of less than about 1000 nm, but greater than about 100 nm, e.g. between about 100 nm and 500 nm, between about 200 nm and about 300 nm, between about 160 nm and about 190 nm, etc.

In certain embodiments, the pharmaceutical composition of the disclosure may comprise spray dried particles having an average diameter of between about 5 μm and about 50 μm in diameter, between about 5 μm and about 30 μm in diameter, between about 5 μm and about 20 μm in diameter, between about 5 μm and about 10 μm in diameter, etc.

In certain embodiments, the pharmaceutical compositions may include a high drug load of tricaprylin, of at least about 20% of the total composition, at least about 25% of the total composition, at least about 30% by weight of the total composition, at least about 40% by weight of the total composition, about 30% by weight of the total composition to about 65% by weight of the total composition, about 30% by weight of the total composition to about 60% by weight of the total composition, about 40% by weight of the total composition to about 50% by weight of the total composition, about 40% by weight of the total composition to about 45% by weight of the total composition, etc.

As used herein, unless other specified, “% by weight” refers to “% by weight of the total composition”.

In certain embodiments, the tricaprylin and/or pharmaceutical compositions may be administered orally. Therapeutically effective amounts of tricaprylin can be any amount or dose sufficient to bring about the desired effect and depend, in part, on the severity and stage of the condition, the size and condition of the patient, as well as other factors readily known to those skilled in the art. The dosages can be given as a single dose, or as several doses, for example, once daily, twice daily, three times daily, etc., as discussed elsewhere herein.

In certain aspects, the tricaprylin may be administered in a pharmaceutical composition comprising a high drug loading of tricaprylin and one or more emulsion forming excipients present at a concentration sufficient to form an emulsion at room temperature. The pharmaceutical compositions may comprise the components in amounts as described herein. In some embodiments, the pharmaceutical compositions may form a stable liquid emulsion.

As described herein, the pharmaceutical compositions of the disclosure may form a liquid emulsion. An emulsion refers to a composition which, when diluted with water or other aqueous medium and gently mixed, yields a stable oil/water emulsion with a mean droplet diameter of less than about 5 μm, but greater than about 100 nm, (e.g., 0.35-1.2 μm) and which is generally polydisperse. Such an emulsion is stable, meaning there is no visibly detectable phase separation and that there is no visibly detectable crystallization.

“Gently mixed” as used herein is understood in the art to refer to the formation of an emulsion by gentle hand (or machine) mixing, such as by repeated inversions on a standard laboratory mixing machine. High shear mixing is not required to form the emulsion. Such emulsion compositions generally emulsify nearly spontaneously when introduced to an aqueous use environment.

As discussed above, the pharmaceutical compositions of the disclosure may form stable emulsions in an aqueous use environment, e.g., in water, pharmaceutically suitable aqueous solution, or when administered in vivo. By way of example, the emulsions may be stable at ambient conditions for at least about 24 hours, at least about one day, at least about 5 days, at least about 10 days, at least about one month, etc. In certain embodiments, the emulsion formed does not phase separate for the duration of stability. In certain embodiments, the emulsions may have a mean droplet diameter of less than about 5 μm, but greater than about 100 nm, (e.g., 0.35-1.2 μm).

In certain embodiments, the emulsion formed may be stable at stomach pH, e.g., at a pH of about 1 to about 3, about 1.2 to 2.9, etc. In certain embodiments, the emulsion formed may be stable at intestinal and/or colon pH, e.g., at a pH of about 5 to about 7, about 5.5 to about 6.9, etc. In certain embodiments, the emulsion formed may begin to break down or phase separate at stomach pH after about 12 to about 1 hour, but does not release the encapsulated tricaprylin until intestinal or colon pH. In this regard, without intending to be limited by theory, in-vitro digestion assays indicate that encapsulated tricaprylin is released from emulsion at intestinal and/or colon pH, which is the primary location of lipid digestion enzymes. In accordance with certain aspects of the disclosure, preferential release of tricaprylin in the intestines and/or colon rather than the stomach may increase bioavailability of the tricaprylin given the location of lipid digestion enzymes in these areas.

In certain aspects of the disclosure, the pharmaceutical compositions provide for preferential release of the high drug loading of tricaprylin in the lower gastrointestinal tract of a user. Without intending to be limited by theory, preferential release of tricaprylin in the lower gastrointestinal tract, including the colon may provide reduced stomach upset and related adverse events as compared to standard administration of non-formulated MCT oil. Further, the improved bioavailability of tricaprylin may generally lead to increased ketone body production in vivo, as compared to standard administration of non-formulated MCT oil.

In certain aspects, the pharmaceutical compositions of the disclosure include one or more emulsion forming excipients. In certain embodiments, the one or more emulsion forming excipients may be any emulsifier capable of forming an emulsion with MCT oil. By way of example, lecithin (e.g., Phospholipon 90G), hydrogenated castor oils including Polyoxyl 40 castor oil (e.g., Kolliphor RH40), caprylate esters, sodium oleate, glycerol, citric acid esters of monoglycerides and diglycerides (e.g., Citrem), monoglycerides and diglycerides of fatty acids including Propylene Glycol Monocaprylate (e.g. Capmul PG-8), and combinations thereof. The emulsion forming excipient(s) may be present in amounts sufficient to provide desired emulsion formation. For example, in certain embodiments, the emulsion forming excipient may be present in an amount of between about 1% and about 10%, between about 1.3% and about 10%, etc., by weight of the total composition.

In certain embodiments, the emulsion forming excipients may include combinations of lecithin, Kolliphor RH40, and caprylate ester emulsifiers. In other embodiments, the emulsion forming excipients may include combinations of lecithin, sodium oleate, and glycerol. In yet other embodiments, the emulsion forming excipients may include Citrem alone or in combination with monoglycerides and diglycerides of fatty acids.

By way of non-limiting example, Table 1 below provides exemplary liquid pharmaceutical formulation attributes and properties.

TABLE 1

| Product Attribute | | Property |
|---|---|---|
| Exemplary Indication(s) | | Migraine, Headache, Neuroinflammation |
| Patient Population | | Young Adults, Adults, Geriatric |
| Dosage Form | | Oil in liquid emulsion, single-dose |
| | | Ready to drink |
| | | 30 mL light protected container |
| Particle size of the emulsion | | 0.35-1.2 um |
| Potency and Impurity | | 90-110%, total impurity less than 5% |
| pH | | Neutral or near Neutral (pH 6-pH 8) |
| Dosing Interval | | Once daily, Twice daily, Three times daily |
| Dose | Primary | 50 g/100 mL (50% w/v MCT) |
| Strength(s) | strengths | |
| | Max/min | To allow for doses of 5 g to 15 g as a |
| | dosing vol. | single dose |
| Dosing | Time and | 30′ after completion of a meal |
| Regimen | duration | |

| Anticipated Formulation | Phospholipon 90G, Kolliphor RH40, |
|---|---|
| Ingredients | Citrem, Monoglycerides, Capmul PG-8, |
| | Sodium Oleate, Glycerol, |
| | flavouring/sweetening agent(s) |

By way of non-limiting example, suitable lecithin useful as an emulsion forming excipient of the disclosure may be derived from any suitable source, e.g., egg or soy. By way of non-limiting example, suitable lecithin may be selected from Soy PC, 95%, Avanti Number 441601; Egg PC, 95%, Avanti Number 131601; etc.

Any suitable monoglyceride or diglyceride of fatty acids may be used as an emulsion forming agent of the disclosure, e.g., citric acid esters of mono and diglycerides of fatty acids (Citrem) E472C; mono and diglycerides of fatty acids E471; etc.

In other embodiments, the pharmaceutical composition may comprise a high drug loading of tricaprylin; at least one surfactant; an adsorbent, and a film forming polymer. The pharmaceutical composition may further include a co-surfactant. In some embodiments, the pharmaceutical composition comprises at least two surfactants. In certain embodiments, the composition is a self-emulsifying, spray dried composition.

In other aspects, the at least one surfactant is selected from polyoxyl hydrogenated castor oil, polyoxyl stearate, polyoxyl hydroxystearate, lecithin, phosphatidylcholine, and combinations thereof. In certain embodiments, the solid composition comprises at least two surfactants, which may be selected from polyoxyl hydrogenated castor oil, polyoxyl stearate, polyoxyl hydroxystearate, lecithin, phosphatidylcholine, and combinations thereof. In certain embodiments, at least one of the at least two surfactants is a polyoxyl hydrogenated castor oil or polyoxyl stearate surfactant. The at least two surfactants may be present in a 2:1 to 1:1 ratio, relative to each other.

In certain aspects, the adsorbent is a silica compound, e.g., colloidal silicon dioxide (AEROSIL®, CAB-O-SIL®), amorphous silica gel (SYLOID®, SYLYSIA®), granulated silicon dioxide (AEROPERL®), silica aerogel, magnesium alumino metasilicates (NEUILIN®), calcium silicate (FLORITE®), and ordered mesoporous silicates.

In certain aspects, the film forming polymer may be polyvinylpyrrolidone (PVP), polyvinylpyrrolidone-vinyl acetate copolymer (PVP-VA), hydroxypropyl methylcellulose (HPMC), hydroxypropyl methylcellulose acetate succinate (HPMCAS), dextrans of varying molecular weights (e.g., 10000, 40000, 70000, 500000, etc.), etc. In certain embodiments, the film forming polymer is PVP or PVP-VA, in other embodiments the film forming polymer is PVP-VA.

Any suitable method for making the pharmaceutical compositions of the disclosure may be used.

The pharmaceutical compositions of the disclosure, in one embodiment, are administered in a dosage required to treat migraine and related headaches (e.g., trigeminal autonomic cephalgia) and headache symptoms. Appropriate dosages may be determined by one of skill in the art. In one embodiment, oral administration of a pharmaceutical composition of the disclosure results in hyperketonemia. Hyperketonemia, in one embodiment, results in ketone bodies being utilized to treat migraine and related headaches (e.g., trigeminal autonomic cephalgia) and headache symptoms.

Administration may be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. Administration may be provided with or without food.

The pharmaceutical compositions provided herein are, in one embodiment, intended for “long term” consumption, sometimes referred to herein as for ‘extended’ periods. “Long term” administration as used herein generally refers to periods in excess of one month. Periods of longer than two, three, or four months comprise one embodiment of the instant invention. Also included are embodiments comprising more extended periods that include longer than 5, 6, 7, 8, 9, or 10 months. Periods in excess of 11 months or 1 year are also included. Longer terms use extending over 1, 2, 3 or more years are also contemplated herein. “Regular basis” as used herein refers to at least weekly, dosing with or consumption of the compositions. More frequent dosing or consumption, such as twice or thrice weekly are included. Also included are regimens that comprise at least once daily consumption. The skilled artisan will appreciate that the blood level of ketone bodies, or a specific ketone body, achieved may be a valuable measure of dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

Administration can be carried out on a regular basis, for example, as part of a treatment regimen in the patient. A treatment regimen may comprise causing the regular ingestion by the patient of a pharmaceutical composition of the disclosure in an amount effective to enhance cognitive function, memory, and behavior in the patient. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a regimen, administration can be multiple times per day. The goal of regular administration is to provide the patient with optimal dose of a pharmaceutical composition of the disclosure, as exemplified herein.

In one embodiment, the composition increases the circulating concentration of at least one type of ketone body in the mammal or patient. In one embodiment, the circulating ketone body is D-beta-hydroxybutyrate. The amount of circulating ketone body can be measured at a number of times post administration, and in one embodiment, is measured at a time predicted to be near the peak concentration in the blood, but can also be measured before or after the predicted peak blood concentration level. Measured amounts at these off-peak times are then optionally adjusted to reflect the predicted level at the predicted peak time. In one embodiment, the predicted peak time is at about two hours. Peak circulating blood level and timing can vary depending on factors known to those of skill in the art, including individual digestive rates, co-ingestion or pre- or post-ingestion of foods, drinks, etc., as known to one of skill in the art. In one embodiment, the peak blood level reached of D-beta-hydroxybutyrate is between about 0.05 millimolar (mM) to about 50 mM. Another way to determine whether blood levels of D-beta-hydroxybutyrate are raised to about 0.05 to about 50 mM is by measurement of D-beta-hydroxybutyrate urinary excretion a range in the range of about 5 mg/dL to about 160 mg/dL. In other embodiments, the peak blood level is raised to about 0.1 to about 50 mM, from about 0.1 to about 20 mM, from about 0.1 to about 10 mM, to about 0.1 to about 5 mM, more preferably raised to about 0.15 to about 2 mM, from about 0.15 to about 0.3 mM, and from about 0.2 to about 5 mM, although variations will necessarily occur depending on the composition and host, for example, as discussed above. In other embodiments, the peak blood level reached of D-beta-hydroxybutyrate will be at least about 0.05 mM, at least about 0.1 mM, at least about 0.15 mM, at least about 0.2 mM, at least about 0.5 mM, at least about 1 mM, at least about 1.5 mM, at least about 2 mM, at least about 2.5 mM, at least about 3 mM, at least about 4 mM, at least about 5 mM, at least about 10 mM, at least about 15 mM, at least about 20 mM, at least about 30 mM, at least about 40 mM, and at least about 50 mM.

Effective amount of dosages of compounds for the inventive compositions, i.e., compounds capable of elevating ketone body concentrations in an amount effective for the treatment of migraine and/or related headache symptoms will be apparent to those skilled in the art. As discussed herein above, such effective amounts can be determined in light of disclosed blood ketone levels.

In one embodiment, the amount of tricaprylin administered is in the range of about 0.05 g/kg/day to about 60 g/kg/day. In other embodiments, the amount of tricaprylin administered is at least 10 g/day, 15 g/day, 20 g/day, 30 g/day, 40 g/day, 50 g/day, 60 g/day. In other embodiments, the dose of tricaprylin will be in the range of about 0.25 g/kg/day to about 20 g/kg/day. In other embodiments, the tricaprylin dose will be in the range of about 0.5 g/kg/day to about 20 g/kg/day. In other embodiments, the dose will be in the range of about 0.1 g/kg/day to about 10 g/kg/day. In other embodiments, the dose of tricaprylin is at least about 0.05 g/kg/day, at least about 0.1 g/kg/day, at least about 0.15 g/kg/day, at least about 0.2 g/kg/day, at least about 0.5 g/kg/day, at least about 1 g/kg/day, at least about 1.5 g/kg/day, at least about 2 g/kg/day, at least about 2.5 g/kg/day, at least about 3 g/kg/day, at least about 4 g/kg/day, at least about 5 g/kg/day, at least about 10 g/kg/day, at least about 15 g/kg/day, at least about 20 g/kg/day, at least about 30 g/kg/day, at least about 40 g/kg/day, and at least about 50 g/kg/day. Administration may be in single or divided doses, e.g., once, twice or three times daily

Convenient unit dosage containers and/or compositions include sachets or containers of spray dried particles, tablets, capsules, lozenges, troches, hard candies, nutritional bars, nutritional drinks, metered sprays, creams, and suppositories, among others. The compositions may be combined with a pharmaceutically acceptable excipient such as gelatin, oil(s), and/or other pharmaceutically active agent(s). For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with other migraine or headache medications (e.g., triptans or CGRP antagonists), or compounds that enhance the efficiency of glucose utilization, and mixtures thereof.

The daily dose of tricaprylin can also be measured in terms of grams of tricaprylin per kg of body weight (BW) of the subject. The daily dose of tricaprylin can range from about 0.01 g/kg to about 10.0 g/kg BW of the subject. Preferably, the daily dose of tricaprylin is from about 0.1 g/kg to about 5 g/kg BW of the subject. More preferably, the daily dose of tricaprylin is from about 0.2 g/kg to about 3 g/kg of the subject. Still more preferably, the daily dose of tricaprylin is from about 0.5 g/kg to about 2 g/kg of the subject.

The following examples are provided for illustrative purposes only and are not intended to limit the scope of the invention.

### EXAMPLES

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### Key Definitions

Migraine defined according to adapted ICHD-3 criteria following industry standard:

Migraine: A headache with or without aura, lasting for ≥30 minutes, with both following (a) and (b) met:
(a) ≥2 of the following headache characteristics:
   Unilateral location;
   Throbbing/pulsatile quality;
   Moderate to severe pain intensity;
   Exacerbated with exercise/physical activity or causing avoidance of routine physical activity;
(b) During headache ≥1 of the following associated symptoms:
   Nausea and/or vomiting;
   Photophobia and phonophobia.

Probable migraine: A headache with or without aura, lasting for ≥30 minutes, but missing 1 of the migraine features.

Migraine Headache Day (MHD): any calendar day on which a migraine headache or probable migraine headache occurs.

Episodic migraine=4-14 MHDs/month; chronic migraine=≥15 MHDs/month.

### Example 1. Tricaprylin in Migraine Patients

This is a randomised, double-blind, placebo-controlled, parallel-group, multi-centre study of tricaprylin as AC-SD-03 compared with placebo for the reduction of migraine in participants with frequent migraine.

Composition of Tricaprylin Oral Powder for Reconstitution, Formulation AC-SD-03, Oral Powder for Reconstitution

TABLE 2
Component

Tricaprylin (40 wt % of composition)
Polyoxyl 40 hydrogenated castor oil (Kolliphor ® RH40)
Glyceryl mono-oleate (Capmul ® GMO-50)
Phospholipon ® 90 G
Polyvinylpyrrolidone (Kollidon ® K30)
Colloidal silicon dioxide (CAB-O-SIL ® M5P)
Sucralose
Acesulfame Potassium
Orange Cream
Purified Water (prior to spray dry)

Alternatively, the following liquid formulations may be given and compared to placebo (90G: Phospholipon 90G; RH140: Kolliphor RH40):

TABLE 3
Liquid Pharmaceutical Composition

50% Tricaprylin, 6% 90G
50% Tricaprylin, 4% 90G, 2% RH40
50% Tricaprylin, 4% 90G, 2% RH40, 2.5% Glycerol
50% Tricaprylin, 2% 90G, 4% RH40
50% Tricaprylin, 6% Kolliphor RH40
50% Tricaprylin, 4% 90G, 2% Capmul PG-8
50% Tricaprylin, 2% 90G, 4% Capmul PG-8
50% Tricaprylin, 6% Capmul PG-8
50% Tricaprylin, 4% Kolliphor RH40, 2% Capmul PG-8
50% Tricaprylin, 2% Kolliphor RH40, 4% Capmul PG-8
50% Tricaprylin, 2% 90G, 2% Kolliphor RH40, 2% Capmul PG-8
50% Tricaprylin, 5.5% 90G, 0.5% RH40
50% Tricaprylin, 5% 90G, 1% RH40
50% Tricaprylin, 5% 90G, 1% RH40, 2.5% Glycerol
50% Tricaprylin, 2.67% 90G, 1.33% RH40
20% Tricaprylin, 1.6% 90G, 0.8% RH40
20% Tricaprylin, 1.6% 90G, 0.8% RH40, 2.5% Glycerol
50% Tricaprylin, 4% 90G, 2% RH40
50% Tricaprylin, 2% 90G, 2% RH40, 2% Capmul PG-8
20% Tricaprylin, 0.8% Citrem (3212), 0.5% monoglyceride (DMG 0291),
50 mM citrate buffer pH 6
20% Tricaprylin, 0.8% Citrem (3212), 0.5% monoglyceride (DMG 0291),
milliQ-H2O pH 6
20% Tricaprylin, 1.0% Citrem (3212), 0.625% monoglyceride (DMG
0291), milliQ-H2O pH 6
20% Tricaprylin, 0.8% Citrem (KV-30E), 0.5% monoglyceride (P(V)S),
50 mM citrate buffer pH 6
20% Tricaprylin, 2.4% Phospholipon 90G, 2.5% Glycerol, 0.5%
Carboxymethylcellulose pH 7
20% Tricaprylin, 2.4% Phospholipon 90G, 2.5% Glycerol, 1% Safflower
oil pH 7
20% Tricaprylin, 2.4% Phospholipon 90G, 2.5% Glycerol, 5% Safflower
oil pH 7
20% Tricaprylin, 2.4% Phospholipon 90G, 0.06% Sodium Oleate, 2.5%
Glycerol, pH 8
20% Tricaprylin, 2.4% Phospholipon 90G, 0.5% Sodium Oleate, 2.5%
Glycerol, pH 8
20% Tricaprylin, 2.4% Phospholipon 90G, 0.2% Kolliphor RH40, 0.5%
Sodium Oleate, 2.5% Glycerol, pH 8
20% Tricaprylin, 1.6% Phospholipon 90G, 0.8% Citrem (3212), 2.5%
Glycerol, pH 7
50% Tricaprylin, 6% 90G pH 7
50% Tricaprylin, 6% 90G, 2.5% Glycerol pH 7
50% Tricaprylin, 6% 90G, 0.15% Oleate pH 7
50% Tricaprylin, 6% 90G, 0.15% Oleate, 2.5% Glycerol pH 7
50% Tricaprylin, 6% 90G, 0.30% Oleate pH 7
50% Tricaprylin, 6% 90G, 0.30% Oleate, 2.5% Glycerol pH 7
20% Tricaprylin, 2.4% 90G pH 7
20% Tricaprylin, 2.4% 90G, 2.5% Glycerol pH 7
20% Tricaprylin, 2.4% 90G, 0.06% Oleate pH 7
20% Tricaprylin, 2.4% 90G, 0.06% Oleate, 2.5% Glycerol pH 7
20% Tricaprylin, 2.4% 90G, 0.12% Oleate pH 7
20% Tricaprylin, 2.4% 90G, 0.06% Oleate, 2.5% Glycerol pH 7
10% Tricaprylin, 1.2% 90G, 2.5% Glycerol, 0.03% a-Tocopherol, 0.03%
Oleate
20% Tricaprylin, 2.4% 90G, 2.5% Glycerol, 0.06% a-Tocopherol, 0.06%
Oleate

Following a screening period of up to 2 weeks, the study duration is 17 weeks including a 4-week baseline measurement period, after which randomisation occurs, a 12-week treatment period and a 1-week safety follow-up period.

The study will be completed in two parts. In Part 1, 54 participants will be randomised to receive tricaprylin as AC-SD-03, or placebo, in a 1:1 ratio. Once 54 participants are randomised, enrolment will be paused until the randomised participants have completed the study. Based on an unblinded analysis of the data from all evaluable patients from Part 1, a decision to proceed to Part 2 (enrolment of additional participants) will be taken. The decision to continue to Part 2 will be made by the Sponsor, based on a number of factors including the one-sided p-value for the analysis of the primary endpoint, ΔMD_{Mth3}, the safety and tolerability profile observed during Part 1, the observed between-subject variability, as well as trial conduct feasibility. The size of Part 2 will be determined by the between-subject variability observed in Part 1, and will be between 80 and 180 participants.

Number of Participants: Part 1: Approximately 135 participants will be screened to achieve 54 randomised participants and 46 evaluable participants (approximately 23 evaluable participants per treatment group). If a decision is made to proceed to Part 2, approximately 200-450 participants will be screened to achieve 78-180 randomised participants and 66-152 evaluable participants in Part 2. The sample size for Part 2 will be determined based on the observed standard deviation (SD) in Part 1.

Investigational Medicinal Products: The formulation AC-SD-03 contains the active compound tricaprylin and excipients, 1:1.5. Thus 75 g AC-SD-03 contains 30 g tricaprylin and 45 g excipients (carrier, emulsifiers and flavouring). The placebo formulation, AC-SD-03P, contains 30 g safflower oil in place of the active compound tricaprylin and identical excipients to the active formulation.

Treatment Groups and Duration: At the Baseline Visit, all participants will receive a sentinel dose of 12.5 g AC-SD-03 (containing 5 g tricaprylin). Those that do not tolerate this sentinel dose will not be eligible to continue. Those that do tolerate the sentinel dose and meet other eligibility criteria, will enter the baseline measurement period of 4 weeks to determine migraine day participant eligibility for the study and to establish baseline data for comparison of endpoints during the study.

After the 4 weeks baseline measurement period, those who record 4-24 migraine days per month will be randomised to AC-SD-03 or placebo (AC-SD-03P) and will titrate from 12.5 g twice a day to 75 g twice a day, over the course of 2 weeks. Participants who have fewer than four or greater than 24 headache days per month in the baseline period will be discontinued from the study. The participant should not be told the number of migraine headache days on which eligibility is based to avoid biased reporting.

Once titration has completed, all participants will continue on the target dose they are on over an additional 10-week maintenance period.

Upon completion of the 12-week double-blind placebo-controlled treatment phase, there will be a 1-week safety follow-up period.

The primary endpoint will be change from baseline in the number of migraine headache days during Month 3 (ΔMD_{Mth3}).

The secondary endpoints are:
Change from baseline in the number of migraine headache days during Month 1 (ΔMD_{Mth1}) and Month 2 (ΔMD_{Mth2}) of treatment and overall, from Months 1-3;
Change from baseline in duration of migraine of moderate to severe intensity during treatment months 1, 2 and 3;
Change from baseline in duration of migraine during treatment months 1, 2 and 3;
The proportion of participants with a 50% reduction from baseline in number of migraine headache days in treatment months 1, 2, and 3;
Change from baseline in monthly acute migraine medicine use during treatment months 1, 2 and 3;
Time to first usage of acute migraine medication;
Change from baseline Headache Impact Test (HIT-6) score at the end of Months 1, 2 and 3;
Incidence of adverse events;
Clinical safety laboratory tests;
Vital signs, ECGs, C-SSRS;

The exploratory objectives are:
Evaluation of treatment of chronic versus episodic migraine headache attacks;
Change from baseline in the number of headache days (migraine and non-migraine headache) during treatment months 1, 2 and 3 (ΔHDMth1, ΔHDMth2 and ΔHDMth3);
Change from baseline in migraine severity during treatment months 1, 2 and 3;
Change from baseline in MIDAS score at the end of Month 1, 2 and 3;
Plasma βHB and other pharmacokinetic parameters;

### Study Results

After 3-months of tricaprylin treatment, a significant reduction of mean migraine frequency (from a mean of 4.1 to 1.4 attacks/month, p<0.001) and duration (from 51.9 to 16.3 hours/month, p<0.001) is anticipated.

### Key Inclusion Criteria:

Age 18 to 70 years;
Frequent (episodic or chronic)* migraine with or without aura, for at least 1 year (per ICHD-3-beta), age at time of onset must be <50 years;
Participants must have 4-24 migraine headaches days per month, as confirmed by the baseline measurement period;
Both episodic and chronic migraine were included (4-24 MHDs), episodic migraine=4-14 MHDs/month; chronic migraine=≥15 MHDs/month;
Use of one allowed migraine prophylactic is permitted if the participant has been on a stable dose for at least 2 months prior to the screening;
Must have failed (no therapeutic response) 1-4 categories of migraine prophylactic treatments;
Tolerates sentinel dose;
Minimum 80% e-Diary Compliance during Baseline period (28 days)

### Key Exclusion Criteria:

History of hemiplegic migraine, cluster headache, or other trigeminal autonomic cephalgia;
Presence of a chronic pain syndrome (other than migraine), such as fibromyalgia;
Use of barbiturates (and/or butalbital-containing analgesics) or opioids (and/or opioid-containing analgesics) for migraine acute treatment ≥4 days per month;
Presence or history of an advanced, severe, progressive, or unstable disease of any type that could interfere with efficacy: or, any active GI conditions, not well controlled by medication e.g. diverticulitis, Crohn's disease, severe GERD, peptic ulcer disease;
Use in the last 3 months of CGRP agents, Botox injections, TENS, cranial nerve blocks, trigger-point injections, acupuncture specifically for migraine, infusion therapy;
Ketogenic diet, low-carb diet, intermittent fasting (including the 5:2 diet);

### Key Protocol Deviations:

Subjects who did not have intended indication (4-24 Migraine Headache Days)—5 in the study;
Note—these subjects were not excluded in the SAP-defined analyses.

### Primary Endpoint and Analysis Sets:

Primary Endpoint—Change from Baseline in Migraine Headache Days during Month 3.
   Note, month 1 and 2 ΔMHDs are calculated analogously, and data from months 1 and 2 are also used in the statistical model for the primary endpoint evaluation.
Analysis Sets—
   Full Analysis Set (FAS): All randomised subjects who received at least one dose of IMP, analysed in accordance with intended treatment arm;
   Safety Set (SAF): Same as above, but analysed according to treatment received;
   Evaluable for Efficacy Set (EES): Subset of the FAS with at least 14/28 diary entries in any post-baseline month;
   Intended Titration Evaluable for Efficacy Set (EEITS): A per-protocol sensitivity analysis set for Protocol Amendment 3. The subset of the EES who received a 21-day titration, and achieved a maximum daily dose no higher than 40 g of Tricaprylin/Placebo between days 16 and 21.
   NOTE: key protocol deviations were not excluded from the above analysis sets.

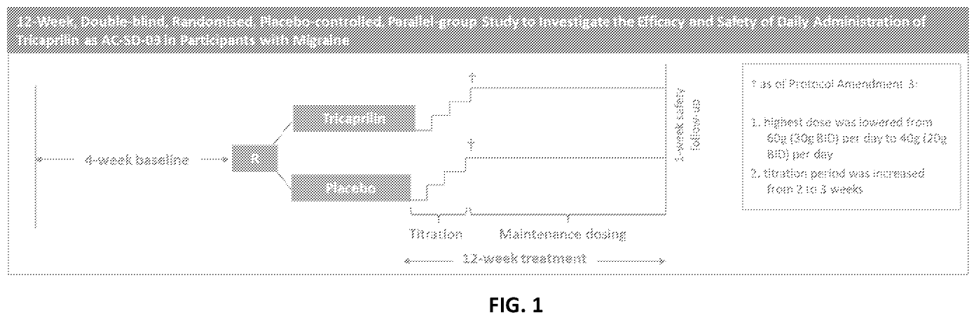

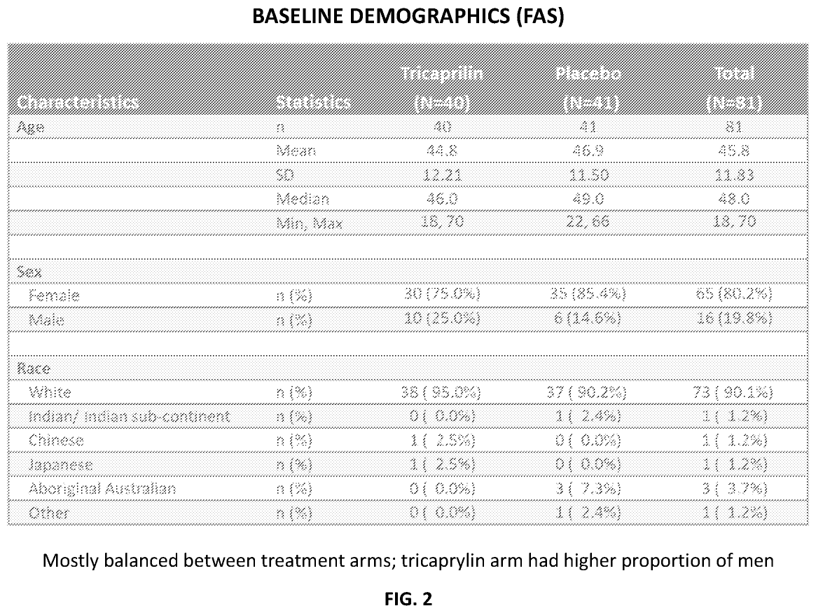

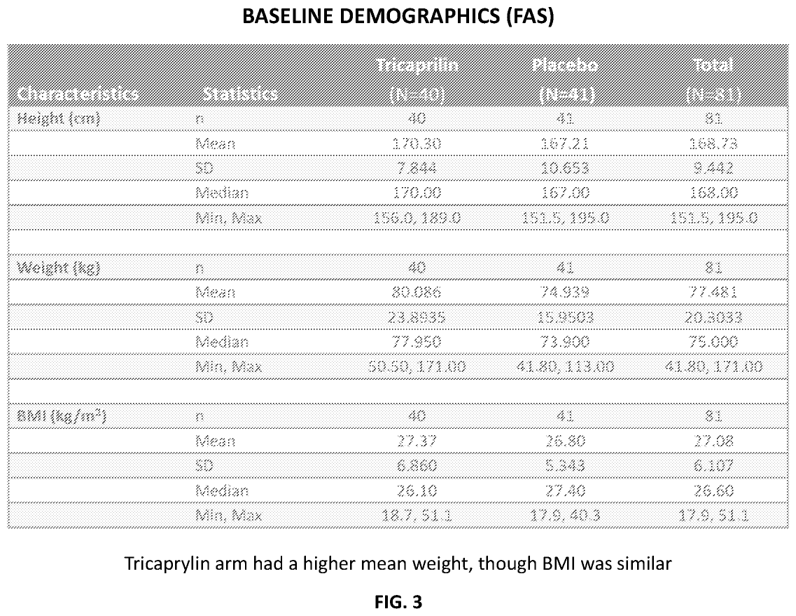

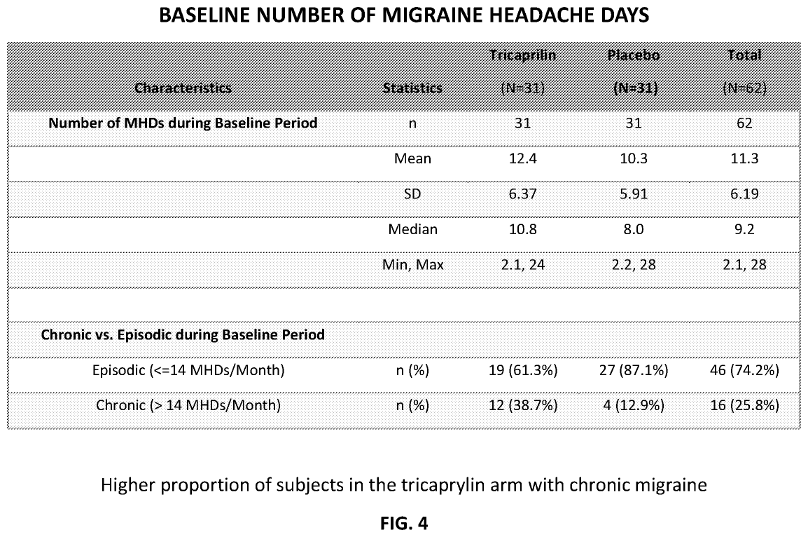

Subjects were enrolled in the study as follows (see FIGS. 2-3 for Baseline Demographics, and FIG. 4 for Baseline MMD, FIG. 1 for dosing and titration protocol scheme):

TABLE 4

| | Tricaprylin | | Placebo | | Total | |
|---|---|---|---|---|---|---|
| | (N = 41) | | (N = 42) | | (N = 83) | |
| Randomised | 41 | | 42 | | 83 | |
| Treated | 40 | | 41 | | 81 | |
| Completed Study Treatment | 22 | (55.0%) | 19 | (46.3%) | 41 | (50.6%) |
| Discontinued treatment prior | 18 | (45.0%) | 22 | (53.7%) | 40 | (49.4%) |
| to completion | | | | | | |
| Primary Reason for | | | | | | |
| Discontinuation: | | | | | | |
| Adverse Event | 12 | (30.0%) | 15 | (36.6%) | 27 | (33.3%) |
| Non-compliance to IMP per | 0 | (0.0%) | 3 | (7.3%) | 3 | (3.7%) |
| protocol | | | | | | |
| Other | 5 | (12.5%) | 3 | (7.3%) | 8 | (9.9%) |
| No reason given | 1 | (2.5%) | 1 | (2.4%) | 2 | (2.5%) |

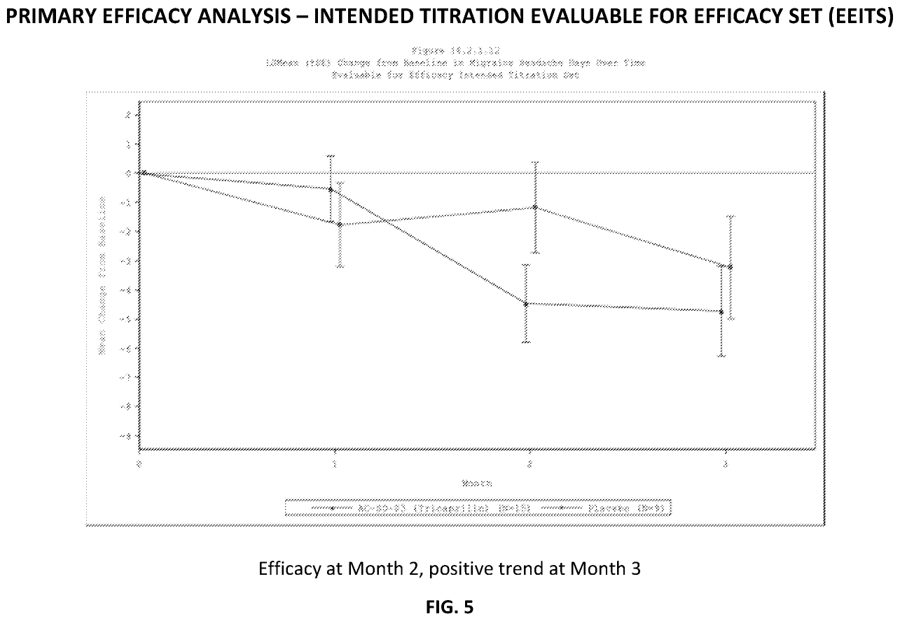

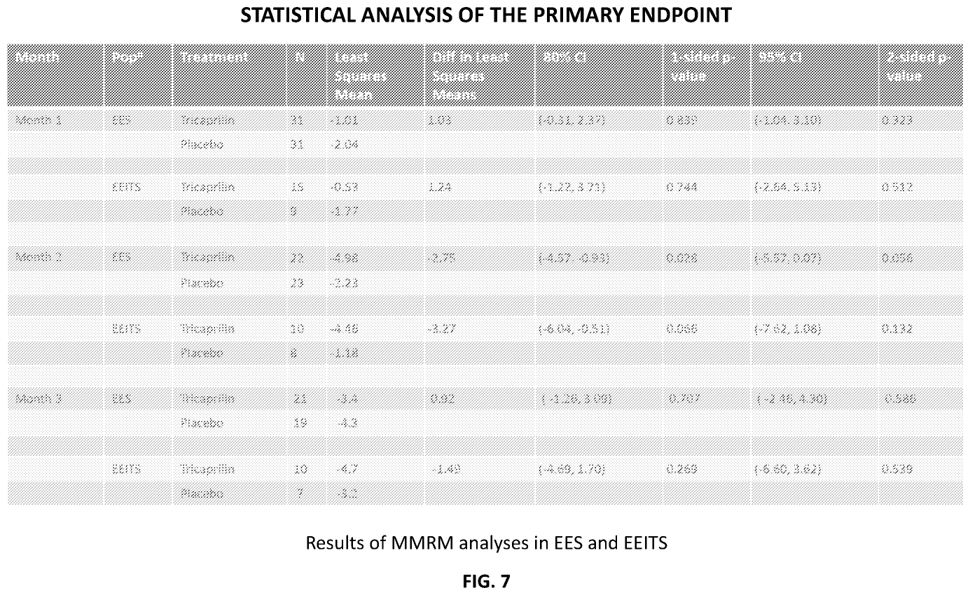

Month 2 results showed a clinically-relevant benefit of tricaprylin (One-sided p=0.066 vs our target of p<0.2). In Month 3, the EEITS showed a positive, clinically-relevant trend approaching the target of p<0.2 (p=0.269). (See FIGS. 5-7)

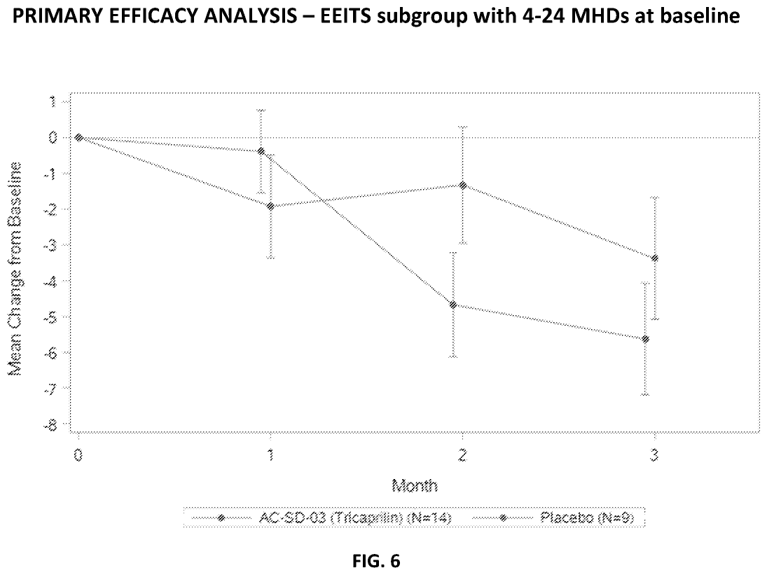

The sensitivity analysis with patients with 4-24 MHD at baseline, in the EEITS shows consistent results with the pre-specified EEITS population. (See FIG. 6)

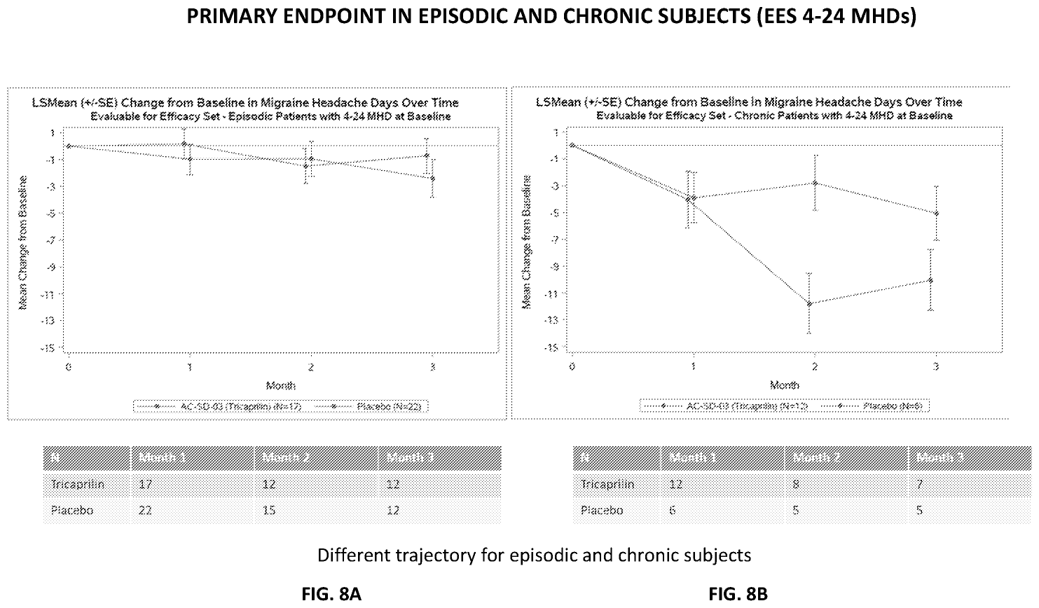

The sub-group analysis revealed a significant interaction for chronic/episodic (p=0.0076). (See FIGS. 8A-8B)

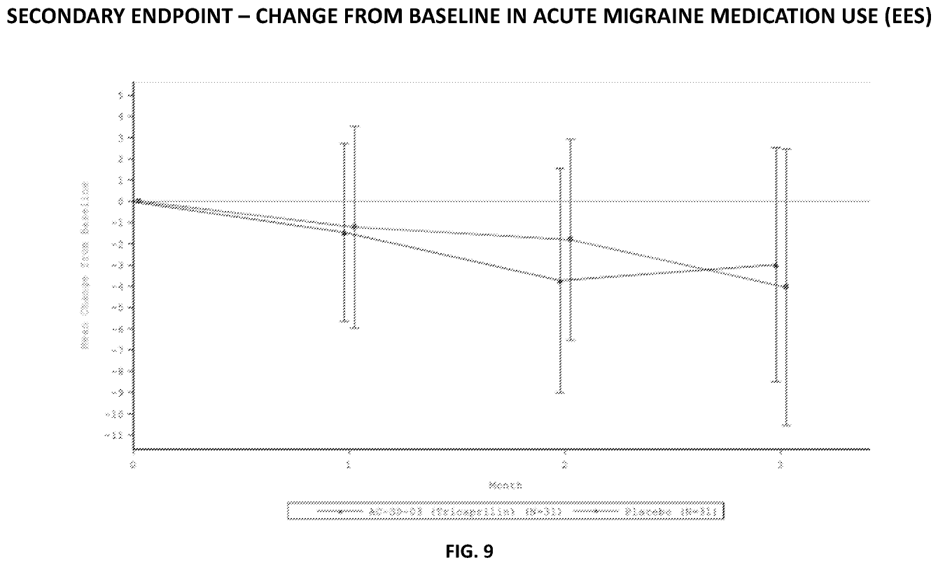

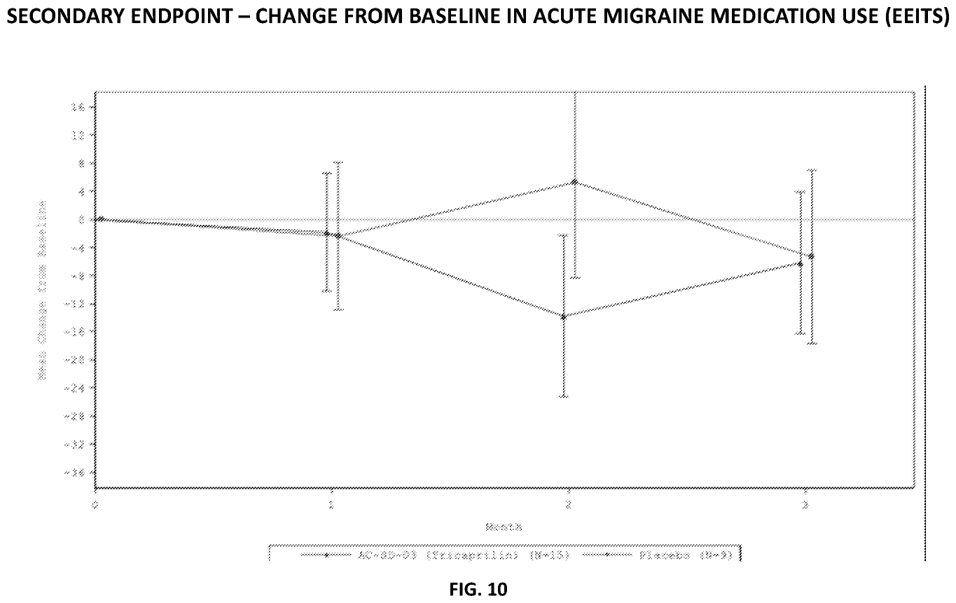

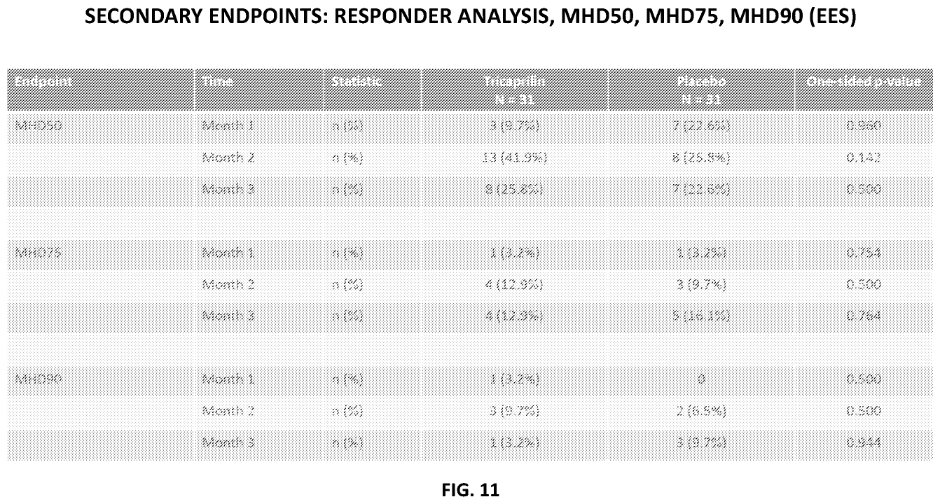

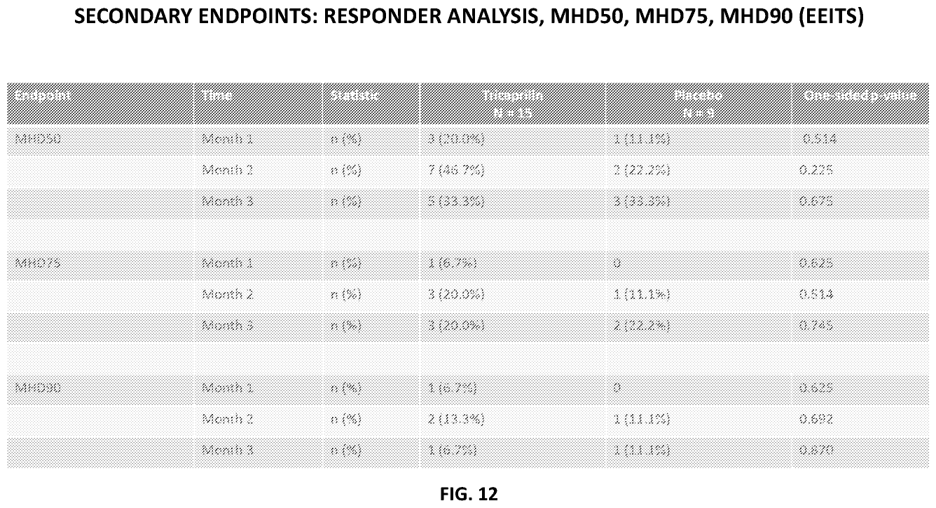

Secondary endpoint analysis, including responder analysis, MHD50, MHD75, MHD90 generally follow the trajectory of the primary endpoint, as illustrated in FIGS. 9-12.

### Example 2: Trigeminal Autonomic Cephalgia Responsive to Tricaprylin

A 67 year old man presented with a history of waxing and waning left hemi-cephalgia over the course of 10 years. His past medical history included bilateral loss of hearing (right worse than left) and past episodes of dizziness leading to a diagnosis of possible Menieres disease. He had hyperlipedemia controlled with a statin. He had some excess weight (BMI 30.8), was a vegetarian and was physically active.

The cephalgia was described as a dull achy non-throbbing pain that when present is continuous over the left side of the face and across the left scalp. The pain appeared to be centered around the eye and was described as if he were being poked in the eye. The pain also involved the left cheek and left scalp. He could not identify any specific trigger to the pain but did note that it was accompanied by tenderness to touch of the scalp on the left side of the head, including sensitivity to hair brushing. When the pain was present he would be aware of it upon awakening in the morning and it would persist throughout the day.

He had no other headache types, and had not experienced episodes of nasal stuffiness, or pupillary changes. He noted occasional excess lacrimation of the left eye. At one point he was investigated for potential ‘sinus problems’ and no sinus condition was diagnosed. Overall the cephalgia was mild and did not interfere with activities of daily living, described more as ‘annoying’ than painful. He self-medicated with occasional paracetamol which provided modest relief. He was intolerant of NSAIDs and had not had a trial of indomethacin.

He had previously had a MRI of the brain which was normal and had been treated with amitriptyline 10 mg which reduced the degree of discomfort partially but led to fatigue and overall the benefit-risk was not felt to be positive so it was discontinued.

Family history included late onset dementia (mother) and hearing loss in a maternal niece. The subject was not suffering from dementia or cognitive disturbance.

The subject began a daily treatment regimen with Axona®, a medical food containing the active ingredient tricaprylin. Shortly after starting daily treatment with Axona®, he noted improvement in his cephalgia and complete clearing of the discomfort after 3 months' ingestion. After discontinuing treatment, the discomfort recurred within a month.

### Discussion

Physical examination was unremarkable other than hearing loss and mild overweight. ESR and other inflammatory markers as well as screening chemistry and haematology labs were normal.

At the time of presentation the headache had recurred to an intensity of 4-6/10 and he was restarted on a course of Axona®, starting with 5 g tricaprylin per day, increasing over 2 weeks to 20 g a day.

Within a month of starting Axona® the maximum pain level had decreased to 3-4/10 and on many days the pain was no longer present, however there was awareness of a ‘numbing sensation’ in the area where the pain had previously been located.

Within 2 months of starting Axona®, all abnormal sensations including scalp sensitivity had cleared.

The Axona® was discontinued after 3 months. Within 6 weeks, the pain had recurred. A third course of Axona® produced the same effects, with gradual reduction and clearing of the pain.

What is claimed is:

1. A method for the treatment or prevention of headache in a subject in need thereof, the method comprising:
administering an effective amount of a composition comprising at least 95% pure tricaprylin to a subject in need thereof.

2. The method of claim 1, wherein the headache is trigeminal autonomic cephalgia.

3. The method of claim 1, wherein the headache is migraine.

4. The method of claim 1, wherein the method is for the treatment or prevention of chronic migraine in a subject, wherein the subject experiences at least 15 migraine headaches in a consecutive 30 day period at baseline.

5. The method of claim 3, wherein the method provides reduction of at least 2 migraine headache days a month from baseline after treatment for at least 2 months.

6. The method of claim 3, wherein the method provides reduction of at least 4 migraine headache days a month from baseline after treatment for at least 2 months.

7. The method of claim 4, wherein the method provides reduction of at least 2 migraine headache days a month from baseline after treatment for at least 2 months.

8. The method of claim 4, wherein the method provides reduction of at least 4 migraine headache days a month from baseline after treatment for at least 2 months.

9. The method of claim 4, wherein the method provides reduction of at least 8 migraine headache days a month from baseline after treatment for at least 2 months.

10. The method of claim 4, wherein the method provides reduction of at least 10 migraine headache days a month from baseline after treatment for at least 2 months.

11. The method of claim 3, wherein the method provides for improvement or amelioration in one or more symptoms of migraine selected from: change from baseline in duration of migraine during treatment months 1, 2 or 3; change from baseline in duration of migraine of moderate to severe intensity during treatment months 1, 2 or 3; the proportion of participants with a 50% reduction from baseline in number of migraine headache days in treatment months 1, 2, or 3; the proportion of participants with a 75% reduction from baseline in number of migraine headache days in treatment months 1, 2, or 3; the proportion of participants with a 90% reduction from baseline in number of migraine headache days in treatment months 1, 2, or 3; change from baseline in monthly acute migraine medicine use during treatment months 1, 2 or 3; change from baseline to time to first usage of acute migraine medication during treatment months 1, 2, or 3; change from baseline Headache Impact Test (HIT-6) score at the end of months 1, 2 or 3; occurrence migraine associated adverse events including aura, nausea, vomiting.

12. The method of claim 4, wherein the method provides for improvement or amelioration in one or more symptoms of migraine selected from: change from baseline in duration of migraine during treatment months 1, 2 or 3; change from baseline in duration of migraine of moderate to severe intensity during treatment months 1, 2 or 3; the proportion of participants with a 50% reduction from baseline in number of migraine headache days in treatment months 1, 2, or 3; the proportion of participants with a 75% reduction from baseline in number of migraine headache days in treatment months 1, 2, or 3; the proportion of participants with a 90% reduction from baseline in number of migraine headache days in treatment months 1, 2, or 3; change from baseline in monthly acute migraine medicine use during treatment months 1, 2 or 3; change from baseline to time to first usage of acute migraine medication during treatment months 1, 2, or 3; change from baseline Headache Impact Test (HIT-6) score at the end of months 1, 2 or 3; occurrence migraine associated adverse events including aura, nausea, vomiting.

13. The method of claim 1, wherein the tricaprylin is administered in an amount ranging from about 10 g/kg/day to about 60 g/kg/day of compound in the composition.

14. The method of claim 1, wherein the composition is administered orally in single or divided doses.

15. The method of claim 1, wherein the composition is administered once, twice, or three times daily.

16. The method of claim 1, wherein the composition comprises at least about 30% by weight of the total composition of tricaprylin, and one or more emulsion forming excipients present at a concentration sufficient to form an emulsion that is stable for at least one month under ambient conditions.

17. The method 16, wherein the one or more emulsion forming excipients of the composition are selected from the group consisting of lecithin, hydrogenated castor oils, caprylate esters, sodium oleate, glycerol, citric acid esters of monoglycerides and diglycerides, monoglycerides and diglycerides of fatty acids including Propylene Glycol Monocaprylate, and combinations thereof

18. The method 16, wherein the tricaprylin is present in the composition in an amount of between about 30% and about 60% by weight of the total composition.

19. The method of claim 16, wherein the one or more emulsion forming excipients are present in the composition in an amount of between about 1% and about 10% by weight of the total composition.

20. The method of claim 16, wherein the stable emulsions exhibits an average particle diameter of less than 0.5 μm for at least one month at ambient conditions, preferable less than 0.3 μm for at least one month at ambient conditions, preferable less than 0.2 μm for at least one month at ambient conditions.
